(19) United States
(12) Patent Application Publication
(10) Pub. No.: US 2022/0370484 A1
(43) Pub. Date: Nov. 24, 2022

(54) NOVEL USE OF NICOTINAMIDE MONONUCLEOTIDE (NMN) AND NICOTINAMIDE RIBOSIDE (NR)

(71) Applicants: Senju Pharmaceutical Co., Ltd., Osaka-shi, Osaka (JP); Kyoto University, Kyoto-shi, Kyoto (JP)

(72) Inventors: Masao Doi, Kyoto-shi, Kyoto (JP); Takeshi Nakajima, Osaka-shi, Osaka (JP); Mamiko Machida, Osaka-shi, Osaka (JP)

(21) Appl. No.: 17/621,114

(22) PCT Filed: Jun. 24, 2020
(86) PCT No.: PCT/JP2020/024916

§ 371 Date: Dec. 20, 2021

(30) Foreign Application Priority Data
Jun. 25, 2019 (JP) 2019-117642

(51) Int. Cl. A61K 31/706; A61P 27/02; A61K 9/00

(52) U.S. Cl. CPC A61K 31/706; A61P 27/02; A61K 9/0048

(57) ABSTRACT

The present disclosure provides a novel use of nicotinamide mononucleotide (NMN) and nicotinamide riboside (NR). The present disclosure provides a composition that includes nicotinamide mononucleotide (NMN) or nicotinamide riboside (NR) for use in treating or preventing diseases, disorders, or symptoms related to activation of Hsd3b6 or a homolog thereof, and a method using the composition. In one embodiment, a composition including NMN or NR for use in improving the function of meibomian glands, and a method using the composition are provided.

### TECHNICAL FIELD

The present disclosure relates to a novel use of nicotinamide mononucleotide (NMN) and nicotinamide riboside (NR). For example, the present disclosure relates to a composition comprising NMN or NR for use in treating or preventing a disease, disorder, or symptom associated with Hsd3b6 or a homolog thereof or a method using the same. In particular, the present disclosure relates to a composition comprising NMN or NR for use in improving a function of a meibomian gland or a method using the same.

### BACKGROUND ART

In view of the suggested possibility of oxidized forms of nicotinamide adenine dinucleotide (NAD+) intermediate metabolites such as nicotinamide riboside (NR) and nicotinamide mononucleotide (NMN), i.e., enzymatic reaction products of nicotinamide phosphoribosyltransferase (NAMPT), exerting an effect of prolonging the lifespan, development thereof as a supplement is ongoing. However, the mechanism of action of the activity of NMN and NR still has not been completely understood.

### SUMMARY OF INVENTION

### Solution to Problem

The present disclosure provides use of nicotinamide mononucleotide (NMN) and/or nicotinamide riboside (NR) or a composition comprising the same for improving the activity of Hsd3b6 or a homolog thereof. Improvement of the activity of Hsd3b6 or a homolog thereof can provide treatment or prevention of a disease, disorder, or symptom associated with the activity of Hsd3b6 or a homolog thereof.

The present disclosure particularly provides use of nicotinamide mononucleotide (NMN) and/or nicotinamide riboside (NR) or a composition comprising the same for improving the function of a meibomian gland. A composition can be characterized by being administered to an eye of a subject. Examples of improvement of a function of a meibomian gland include an increase in the number of meibomian gland acinar cells, increase in lipid secretion from a meibomian gland, and the like. In one embodiment, a composition for use in treating or preventing a disease accompanied by meibomian gland dysfunction can be provided. In another embodiment, the composition of the present disclosure exhibits an effect of improving meibomian gland tissue atrophy and can elevate synthase activity of a steroid that affects a lipid secreted from a meibomian gland. Since the composition of the present disclosure can normalize composition of lipids secreted from a meibomian gland, it is understood that lipids in the meibomian gland can be normalized. Furthermore, the composition of the present disclosure can promote production of testosterone, which is known to promote lipid production of a meibomian gland. Since sex hormones such as testosterone affects the lipid production of meibomian glands, it is understood that the composition of the present disclosure can normalize lipid component composition in meibomian gland tissue.

For example, the following items are provided in preferred embodiments of the present disclosure.

### (Item A1)

A composition comprising nicotinamide mononucleotide (NMN) and/or nicotinamide riboside (NR) for use in improving activity of Hsd3b6 or a homolog thereof.

### (Item A1-2)

A composition comprising nicotinamide mononucleotide (NMN) and/or nicotinamide riboside (NR) for use in treating or preventing a disease, disorder, or symptom associated with activity of Hsd3b6 or a homolog thereof.

### (Item A1-3)

The composition of any of the preceding items, wherein the disease associated with activity of Hsd3b6 or a homolog thereof is selected from the group consisting of meibomian gland dysfunction, diseases due to reduced steroid hormone (e.g., testosterone, estrogen, or the like), postmenopausal syndrome, osteoporosis, life style diseases, androgen deficiency in aging male, and myocardial infarction.

### (Item A2)

The composition of any of the preceding items, wherein the composition is administered to an eye of a subject.

### (Item A3)

A composition comprising nicotinamide mononucleotide (NMN) and/or nicotinamide riboside (NR) for use in improving a function of a meibomian gland.

### (Item A4)

The composition of any of the preceding items for use in increasing a number of meibomian gland acinar cells.

### (Item A5)

The composition of any of the preceding items for use in increasing lipid secretion from a meibomian gland.

### (Item A6)

The composition of any of the preceding items for use in treating or preventing a disease accompanied by dysfunction of a meibomian gland.

### (Item A6-1)

A composition for use in treating or preventing meibomian gland dysfunction comprising nicotinamide mononucleotide (NMN) and/or nicotinamide riboside (NR).

### (Item A6-2)

The composition of any of the preceding items, wherein the meibomian gland dysfunction is accompanied by reduced meibum secretion.

### (Item A6-3)

The composition of any of the preceding items, wherein the meibomian gland dysfunction is accompanied by an inflammatory disease.

### (Item A6-4)

The composition of any of the preceding items, wherein the inflammatory disease comprises at least one selected from the group consisting of meibomitis, superficial (punctate) keratitis, and blepharitis.

### (Item A6-5)

The composition of any of the preceding items, wherein the meibomian gland dysfunction is accompanied by excessive lipid accumulation in a duct.

### (Item A6-6)

The composition of any of the preceding items, wherein the meibomian gland dysfunction is accompanied by ocular discomfort, sensation of a foreign object, and/or sense of pressure.

### (Item A7)

The composition of any of the preceding items, wherein the disease accompanied by meibomian gland dysfunction is meibomitis, posterior blepharitis, keratitis, conjunctivitis, meibomitis-related keratoconjunctivitis, ocular pemphigoid, Sjogren's syndrome, Stevens-Johnson syndrome, graft-versus-host disease (GVHD), and visual function disorder.

### (Item B1)

A method of improving activity of Hsd3b6 or a homolog thereof in a subject or treating or preventing a disease, disorder, or symptom associated with activity of Hsd3b6 or a homolog thereof.

### (Item B2)

The method of item B1, comprising administering nicotinamide mononucleotide (NMN) and/or nicotinamide riboside (NR) to the subject.

### (Item B3)

The method of item B1 or B2, further comprising measuring activity of Hsd3b6 or a homolog thereof in the subject.

### (Item B4)

The method of any of items B1 to B3, comprising administering NMN and/or NR when the activity of Hsd3b6 or a homolog thereof in the subject is measured and the activity is diminished.

### (Item C1)

A method of improving a function of a meibomian gland of a subject or treating or preventing a disease, disorder, or symptom accompanied by dysfunction of a meibomian gland.

### (Item C2)

The method of item C1, comprising administering nicotinamide mononucleotide (NMN) and/or nicotinamide riboside (NR) to the subject.

### (Item C3)

The method of item C1 or C2, further comprising inspecting a meibomian gland function of the subject.

### (Item C4)

The method of any of items C1 to C3, comprising administering NMN and/or NR when a meibomian gland function of the subject is inspected and the function is diminished.

### (Item C5)

The method of item C4, wherein the meibomian gland function is inspected by a slit-lamp examination, Meibography, Confocal microscopy, DR-1, mucocutaneous junction, meibometer, Lipid chemistry, amount of evaporation, BUT, or a subjective symptom questionnaire.

### (Item C6)

The method of any of items C1 to C5, wherein the disease, disorder, or symptom accompanied by dysfunction of a meibomian gland is selected from the group consisting of meibomitis, posterior blepharitis, keratitis, conjunctivitis, meibomitis-related keratoconjunctivitis, ocular pemphigoid, Sjogren's syndrome, Stevens-Johnson syndrome, graft-versus-host disease (GVHD), and visual function disorder.

### (Item C7)

The method of any of items C1 to C6 for increasing a number of meibomian gland acinar cells.

### (Item C8)

The method of any of items C1 to C6 for increasing lipid secretion from a meibomian gland.

### (Item C9)

The method of any of items C1 to C6 for enhancing testosterone generation in a meibomian gland.

### (Item D1)

Use of nicotinamide mononucleotide (NMN) and/or nicotinamide riboside (NR) for manufacturing a composition that improves a function of a meibomian gland.

### (Item D2)

The use of item D1, wherein the composition is for increasing a number of meibomian gland acinar cells.

### (Item D3)

The use of item D1, wherein the composition is for increasing lipid secretion from a meibomian gland.

### (Item D4)

The use of any one of items D1 to D3, wherein the composition is for treating or preventing a disease accompanied by dysfunction of a meibomian gland.

### (Item D5)

The use of item D4, wherein the disease accompanied by dysfunction of a meibomian gland is selected from the group consisting of meibomitis, posterior blepharitis, keratitis, conjunctivitis, meibomitis-related keratoconjunctivitis, ocular pemphigoid, Sjogren's syndrome, Stevens-Johnson syndrome, graft-versus-host disease (GVHD), and visual function disorder.

### (Item D6)

The use of any one of items D1 to D5, wherein the composition is for enhancing testosterone generation in a meibomian gland.

### (Item E1)

Use of nicotinamide mononucleotide (NMN) and/or nicotinamide riboside (NR) for manufacturing a composition for use in treating or preventing meibomian gland dysfunction.

### (Item E2)

The use of item E1, wherein the meibomian gland dysfunction is accompanied by reduced meibum secretion.

### (Item E3)

The use of item E1, wherein the meibomian gland dysfunction is accompanied by an inflammatory disease.

### (Item E4)

The use of item E3, wherein the inflammatory disease comprises at least one selected from the group consisting of meibomitis, superficial (punctate) keratitis, and blepharitis.

### (Item E5)

The use of item E1, wherein the meibomian gland dysfunction is accompanied by excessive lipid accumulation in a duct.

### (Item E6)

The use of item E1, wherein the meibomian gland dysfunction is accompanied by ocular discomfort, sensation of a foreign object, and/or sense of pressure.

### (Item F1)

Nicotinamide mononucleotide (NMN) and/or nicotinamide riboside (NR) for use in improving activity of Hsd3b6 or a homolog thereof in a subject or treating or preventing a disease, disorder, or symptom associated with activity of Hsd3b6 or a homolog thereof.

### (Item G1)

Nicotinamide mononucleotide (NMN) and/or nicotinamide riboside (NR) for use in improving a function of a meibomian gland or treating or preventing a disease, disorder, or symptom accompanied by dysfunction of a meibomian gland.

### (Item G2)

The nicotinamide mononucleotide (NMN) and/or nicotinamide riboside (NR) of item G1, wherein the disease, disorder, or symptom accompanied by dysfunction of a meibomian gland is selected from the group consisting of meibomitis, posterior blepharitis, keratitis, conjunctivitis, meibomitis-related keratoconjunctivitis, ocular pemphigoid, Sjogren's syndrome, Stevens-Johnson syndrome, graft-versus-host disease (GVHD), and visual function disorder.

### (Item H1)

A composition for use in enhancing testosterone generation in a meibomian gland, comprising nicotinamide mononucleotide (NMN) and/or nicotinamide riboside (NR).

### (Item H2)

The composition of item H1, wherein the composition is administered to an eye of a subject.

### (Item I1)

A method for use in enhancing testosterone generation in a meibomian gland of a subject.

### (Item I2)

The method of item I1, comprising administering nicotinamide mononucleotide (NMN) and/or nicotinamide riboside (NR) to the subject.

### (Item J1)

Nicotinamide mononucleotide (NMN) and/or nicotinamide riboside (NR) for enhancing testosterone generation in a meibomian gland.

### (Item J2)

The nicotinamide mononucleotide (NMN) and/or nicotinamide riboside (NR) of item J1, which is administered to an eye of a subject.

### (Item K1)

Use of nicotinamide mononucleotide (NMN) and/or nicotinamide riboside (NR) for the manufacture of a medicament for enhancing testosterone generation in a meibomian gland.

### (Item K2)

The use of item K1, characterized by administration into an eye of a subject.

The present disclosure is intended so that one or more of the features described above can be provided not only as the explicitly disclosed combinations, but also as other combinations thereof. Additional embodiments and advantages of the present disclosure are recognized by those skilled in the art by reading and understanding the following detailed description as needed.

### Advantageous Effects of Invention

The present disclosure provides a composition for use in improving activity of Hsd3b6 or a homolog thereof, or treating or preventing a disease, disorder, or symptom associated with activity of Hsd3b6 or a homolog thereof, or a method therefor. In particular, the present disclosure can provide a composition for use in improving a function of a meibomian gland or a method therefor.

### BRIEF DESCRIPTION OF DRAWINGS

FIG. 1 is a diagram showing atrophy of meibomian gland tissue associated with aging. At the left and center are pictures of stained eye lids of 6 month old and 24 month old mice, respectively. The right side is a bar graph that compares the mean stained areas for 6 month old and 24 month old mice.

FIG. 2 is a diagram showing the change in meibomian gland tissue of a 2 month old Hsd3b6 knockout (KO) mouse (size of meibomian gland and number of secretory ducts). The top row shows results for male mice and the bottom row shows results for female mice.

FIG. 3 is a diagram showing the change in Hsd3b6 enzymatic activity and proliferative activity of meibomian gland basal cells in a meibomian gland due to eye drop instillation of NMN or NR for 2 weeks into 1.5 year old wild-type mice.

FIG. 4 is a diagram showing the effect of NMN or NR eye drop instillation for 3 months into 1.75 year old wild-type mice on meibomian gland tissue. The figure shows representative pictures from staining eye lids of the left eye (untreated) and right eye (eye drop instillation) and a graph showing the stained area in each individual (right eye/left eye).

FIG. 5 is a diagram showing the testosterone content in meibomian gland tissue after castration and after sham surgery on 2 month old wild-type (WT) and Hsd3b6 knockout (KO) male mice.

### DESCRIPTION OF EMBODIMENTS

The present disclosure is described hereinafter. Throughout the entire specification, a singular expression should be understood as encompassing the concept thereof in the plural form, unless specifically noted otherwise. Thus, singular articles (e.g., “a”, “an”, “the”, and the like in the case of English) should also be understood as encompassing the concept thereof in the plural form, unless specifically noted otherwise. The terms used herein should also be understood as being used in the meaning that is commonly used in the art, unless specifically noted otherwise. Thus, unless defined otherwise, all terminologies and scientific technical terms that are used herein have the same meaning as the general understanding of those skilled in the art to which the present disclosure pertains. In case of a contradiction, the present specification (including the definitions) takes precedence.

### Definitions

As used herein, “about” refers to a range of ±10% from the numerical value that is described subsequent to “about”, unless noted otherwise.

As used herein, “subject” refers to the target of administration (transplantation) of a medicament or method for the treatment and prophylaxis of the present disclosure. Examples of subjects include mammals (e.g., humans, mice, rats, hamsters, rabbits, cats, dogs, cows, horses, sheep, monkeys, and the like), but primates are preferable and humans are particularly preferable.

As used herein, “or” is used when “at least one or more” of the listed matters in the sentence can be employed. When explicitly described herein as “within the range” of “two values”, the range also includes the two values themselves.

As used herein, “therapy (treatment)” refers to healing of a disease or a symptom, or suppression of a symptom. “Prevention (prophylaxis)” refers to the prevention of expression of a disease or a symptom in advance. This concept also encompasses delaying the expression of a disease or symptom and minimizing the expression of a disease or symptom.

As used herein, “derivative” refers to a compound that has a core structure that is the same or similar to that of a parent compound, but has a chemical or physical modification such as a different functional group or an additional functional group. A derivative has the same or similar biological activity as the parent compound.

As used herein, “pharmaceutically acceptable salt” refers to an inorganic or organic acid addition salt of the compound of the present disclosure that is relatively non-toxic. These salts can be prepared temporarily during the final isolation and purification of a compound, or by reacting the purified compound in its free base form thereof separately with a suitable organic or inorganic salt, and isolating a salt thus formed.

Examples of pharmaceutically acceptable basic salts of the compound of the present disclosure include alkali metal salts such as sodium salts and potassium salts; alkaline earth metal salts such as calcium salts and magnesium salts; ammonium salts; aliphatic amine salts such as trimethylamine salts, triethylamine salts, dicyclohexylamine salts, ethanolamine salts, diethanolamine salts, triethanolamine salts, procaine salts, meglumine salts, diethanolamine salts, and ethylenediamine salts; aralkylamine salts such as N,N-dibenzylethylenediamine and benetamine salts; heterocyclic aromatic amine salts such as pyridine salts, picoline salts, quinoline salts, and isoquinoline salts; quaternary ammonium salts such as tetramethylammonium salts, tetraethylammonium salt, benzyltrimethylammonium salts, benzyltriethylammonium salts, benzyltributylammonium salts, methyltrioctylammonium salts, and tetrabutylammonium salts; basic amino acid salts such as arginine salts and lysine salts; and the like.

Examples of pharmaceutically acceptable acidic salts of the compound of the present disclosure include inorganic acid salts such as hydrochlorides, sulfates, nitrates, phosphates, carbonates, hydrogen carbonates, and perchlorates; organic acid salts such as acetates, propionates, lactates, maleates, fumarates, tartrates, malates, citrates, and ascorbates; sulfonates such as methanesulfonates, isethionates, benzenesulfonates, and p-toluenesulfonates; acidic amino acids such as aspartates and glutamates; and the like.

As used herein, “solvate” refers to a solvate of the compound of the present disclosure or a pharmaceutically acceptable salt thereof, encompassing, for example, a solvate of an organic solvent (e.g., alcohol (ethanol or the like)-ate), hydrate, and the like. When forming a hydrate, this can be coordinated with any number of water molecules. Examples of hydrates include monohydrates, dihydrates, and the like.

As used herein, “homolog” refers to a protein or gene having an amino acid sequence or a base sequence derived from the same ancestor. A homolog from speciation is referred to as an ortholog. A homolog newly created by genetic duplication in an organism species is referred to as a paralog.

(Active Ingredient)

Nicotinamide mononucleotide (NMN) is a compound with the following structure

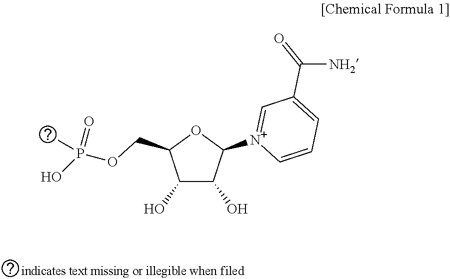

which is a nucleotide derived from nicotinamide and ribose. It is known as a biochemical progenitor of NAD+(nicotinamide adenine dinucleotide).

Nicotinamide riboside (NR) is a compound with the following structure

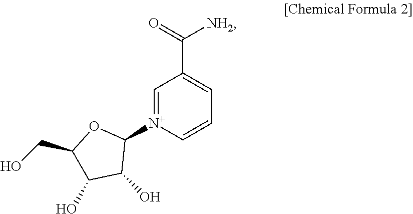 [SMILES: NC(=O)c1ccc[n+]([C@@H]2O[C@H](CO)[C@@H](O)[C@H]2O)c1]

which is a conjugate of ribose and nicotinamide. This can be considered as NMN with a nucleotide replaced with ribose.

The present disclosure can provide a composition comprising NMN and/or NR, or a method including the use (e.g., administration) of NMN and/or NR. NMN and/or NR may be used as a derivative thereof and/or a pharmaceutically acceptable salt thereof. The Examples herein show that NMN and NR can improve Hsd3b6 enzymatic activity (or enzymatic activity of a homolog thereof such as HSD3B1). The Examples herein also show that NMN and NR can restore meibomian gland tissue. In one embodiment, a composition can comprise NMN. In one embodiment, a composition can comprise NR.

NMN and/or NR can be used in combination with an additional active ingredient. Examples of additional active ingredient include, but are not limited to, an anti-inflammatory agent, antimicrobial drug, nutritional supplement, antioxidant, and the like.

(Disease)

The present disclosure can provide a composition for use in or method for improving activity of Hsd3b6 or a homolog thereof with NMN and NR. Hsd3b6 is directly controlled by the circadian clock, and a product of this gene is an important element of an aldosterone production pathway. In addition, a composition for use in or method for treating or preventing a disease, disorder, or symptom associated with activity of Hsd3b6 or a homolog thereof can be provided. Examples of the disease associated with activity of Hsd3b6 or a homolog thereof include, but are not limited to, meibomian gland dysfunction, diseases due to reduced steroid hormone (e.g., testosterone, estrogen, or the like), postmenopausal syndrome, osteoporosis, life style diseases, androgen deficiency in aging male, myocardial infarction, and the like.

In particular, the present disclosure can provide a composition for use in or method for improving a function of a meibomian gland with NMN and NR. A meibomian gland is one of the sebaceous glands that are at the edge of an eyelid. A meibomian gland prevents evaporation of the tear film of an eye, prevents tears from falling onto the cheek, and secretes an oily substance (sebum) which functions to seal the inside of a closed eyelid. A meibomian gland is also known as a tarsal gland. Meibomian gland dysfunction (MGD) is defined as being accompanied by chronic ocular discomfort while having diffuse abnormality in the function thereof. The cause thereof is understood to be meibomian lipid denaturation or stagnation, chronic infection, or hyperkeratosis of inflamed ductal epithelium. The Examples of the present disclosure demonstrate that NMN and NR can restore the Hsd3b6 activity in a meibomian gland, and can increase the size of a meibomian gland. Examples of improvement of a function of a meibomian gland include an increase in the number of meibomian gland acinar cells, increase in lipid secretion from a meibomian gland, and treatment or prevention of a disease accompanied by dysfunction of a meibomian gland.

One embodiment of the present disclosure can provide a composition for use in or method for treating or preventing a disease accompanied by dysfunction of a meibomian gland with NMN and NR. For example, a composition for use in treating or preventing meibomian gland dysfunction, comprising nicotinamide mononucleotide (NMN) and/or nicotinamide riboside (NR) can be provided. Meibomian gland dysfunction can be accompanied by reduced meibum secretion. Meibomian gland dysfunction can be accompanied by an inflammatory disease. The inflammatory disease is, for example, at least one selected from the group consisting of meibomitis, superficial (punctate) keratitis, and blepharitis. Meibomian gland dysfunction can be accompanied by excessive lipid accumulation in a duct. Meibomian gland dysfunction can be accompanied by ocular discomfort, sensation of a foreign object, and/or sense of pressure. For example, the disease accompanied by meibomian gland dysfunction can be, but is not limited to, a disease selected from the group consisting of meibomitis, posterior blepharitis, keratitis, conjunctivitis, meibomitis-related keratoconjunctivitis, visual function disorder, androgen deficiency, atopic dermatitis, prostatic hyperplasia, ocular pemphigoid, discoid lupus (erythematosus), ectodermal dysplasia syndrome (anhidrotic ectodermal dysplasia), bone marrow transplant, hypertension, menopause, Parkinson's disease, plaque psoriasis, rosacea, Sjogren's syndrome, Stevens-Johnson syndrome, toxic epidermal necrolysis, Tuner syndrome, graft-versus-host disease (GVHD), and side effects due to drug therapy (retinoid, anti-androgen, antidepressant, antihistamine drug, or postmenopausal estrogen therapy). It can be understood that the composition of the present disclosure can improve the meibomian gland function itself so that the composition can be effectively used on any of the diseases described above.

Another embodiment of the present disclosure can provide a composition for use in or method for normalizing composition of lipids secreted from a meibomian gland and normalizing lipids in the meibomian gland. Since the composition of the present disclosure exhibits an effect of improving meibomian gland tissue atrophy and enhances synthase activity of steroid that affects lipids secreted from a meibomian gland, it is understood that the composition contributes to normalization of lipids in a meibomian gland. The method of measuring a lipid component in a meibomian gland is not particularly limited. Various methods that are well known in the art can be used, as long as the method can evaluate a lipid component in a meibomian gland.

In one embodiment of the present disclosure, the composition of the present disclosure elevates Hsd3b6 enzymatic activity. Thus, production of testosterone, which is known to promote lipid production of a meibomian gland, can be promoted. Since sex hormones such as testosterone affect lipid production in a meibomian gland, it is understood that the composition of the present disclosure contributes to normalization of lipid component composition in meibomian gland tissue by promoting production of sex hormones including testosterone via elevation of Hsd3b6 enzymatic activity.

(Formulation)

The composition of the present disclosure can be formulated into a suitable dosage form. For example, the composition of the present disclosure, when used as an ophthalmic composition, can be provided as an ophthalmic injection, ophthalmic ointment, eye drops, or ophthalmic perfusate. The composition can be formulated into any dosage form such as aerosol, liquid agent, extract, elixir, capsule, granule, pill, ointment, powder, tablet, solution, suspension, or emulsion. The composition can comprise any pharmaceutically acceptable additive and/or excipient that is known in the art. Examples of additives include, but are not limited to, tonicity adjusting agent, buffer, preservative, cosolvent, and thickener. For example, an ophthalmic composition can be provided in a form of a liquid agent prepared by dissolving an active ingredient into an aqueous solvent (e.g., water).

The composition of the present disclosure can be administered through any suitable route determined by those skilled in the art. The composition can be formulated to be suitable for administration through a route of administration selected from, but is not limited to, ocular injection, topical application (including application to an eye), eye drop, intravenous injection, intravenous drip, oral administration, parenteral administration, transdermal administration, and the like.

Examples of isotonizing agents include saccharides such as glucose, trehalose, lactose, fructose, mannitol, xylitol, and sorbitol, multivalent alcohols such as glycerol, polyethylene glycol, and propylene glycol, inorganic salts such as sodium chloride, potassium chloride, and calcium chloride, and the like. The amount thereof is preferably 0 to 5% by weight with respect to the entire amount of the composition.

Examples of chelating agents include edentates such as disodium edetate, disodium calcium edetate, trisodium edetate, tetrasodium edetate, and calcium edetate, ethylenediamine tetraacetate, nitrilotriacetic acid or a salt thereof, sodium hexametaphosphate, citric acid, and the like. The amount thereof is preferably 0 to 0.2% by weight with respect to the entire amount of the composition.

Examples of stabilizers include sodium hydrogen sulfite and the like. The amount thereof is preferably 0 to 1% by weight with respect to the entire amount of the composition.

Examples of pH modifiers include acids such as hydrochloric acid, carbonic acid, acetic acid, and citric acid, as well as alkali metal hydroxides such as sodium hydroxide and potassium hydroxide, alkali metal carbonates or bicarbonates such as sodium carbonate, alkali metal acetate such as sodium acetate, alkali metal citrate such as sodium citrate, bases such as trometamol, and the like. The amount thereof is preferably 0 to 20% by weight with respect to the entire amount of the composition.

Examples of preservatives include sorbic acid, potassium sorbate, parahydroxybenzoate esters such as methyl parahydroxybenzoate, ethyl parahydroxybenzoate, propyl parahydroxybenzoate, and butyl parahydroxybenzoate, quaternary ammonium salts such as chlorhexidine gluconate, benzalkonium chloride, benzethonium chloride, and cetylpyridinium chloride, alkylpolyaminoethylglycine, chlorobutanol, polyquad, polyhexamethylene biguanide, chlorhexidine, and the like. The amount thereof is preferably 0 to 0.2% by weight with respect to the entire amount of the composition.

Examples of antioxidants include sodium hydrogen sulfite, dried sodium sulfite, sodium pyrosulfite, concentrated mixed tocopherol, and the like. The amount thereof is preferably 0 to 0.4% by weight with respect to the entire amount of the composition.

Examples of solubilizing agents include sodium benzoate, glycerin, D-sorbitol, glucose, propylene glycol, hydroxypropyl methylcellulose, polyvinylpyrrolidone, macrogol, D-mannitol, and the like. The amount thereof is preferably 0 to 3% by weight with respect to the entire amount of the composition.

Examples of thickening agents include polyethylene glycol, methyl cellulose, ethyl cellulose, carmellose sodium, xanthan gum, sodium chondroitin sulfate, hydroxyethyl cellulose, hydroxypropyl cellulose, hydroxypropyl methylcellulose, polyvinylpyrrolidone, polyvinyl alcohol, and the like. The amount thereof is desirably 0 to 70% by weight with respect to the entire amount of the composition.

Eye drops can be prepared by, for example, by dissolving or suspending the desired component described above in an aqueous solvent such as sterile purified water, saline, or buffer (e.g., borate buffer, phosphate buffer, etc.) or a non-aqueous solvent such as vegetable oil such as cottonseed oil, soybean oil, sesame oil, or peanut oil, adjusting the osmotic pressure to predetermined osmotic pressure, and applying sterilization such as mechanical sterilization.

An ophthalmic ointment can comprise an ointment base in addition to the various components described above upon preparation. Examples of the ointment base include, but are not particularly limited to, oily bases such as petroleum jelly, liquid paraffin, and polyethylene, emulsion bases prepared from emulsifying an oil phase and an aqueous phase with a surfactant or the like, water soluble bases consisting of hydroxypropyl methylcellulose, carboxymethyl cellulose, polyethylene glycol, and the like.

The composition, therapeutic agent, or prophylactic agent of the present disclosure can be provided as a kit. In a specific embodiment, the present disclosure provides a drug pack or kit comprising one or more containers filled with one or more components of the composition or medicament of the present disclosure. Optionally, information indicating approval for manufacture, use, or sale for human administration by a government agency regulating the manufacture, use, or sale of drugs or biological products can be displayed on such containers in a form specified by the government agency.

As used herein, “kit” refers to a unit providing parts to be provided (e.g., therapeutic drug, prophylactic drug, each component thereof, user manual, and the like) which are generally separated into two or more segments. Such a kit form is preferred when providing a composition, which should not be provided in a mixed state for stability or the like and is preferably used by mixing immediately prior to use. Such a kit advantageously comprises an instruction or user manual describing how the provided parts (e.g., therapeutic drug or prophylactic drug) are used or how the reagents should be processed. When a kit herein is used as a reagent kit, the kit generally comprises an instruction or the like describing the method of use of the therapeutic agent, prophylactic agent, or the like.

As used herein, “instruction” is a document with an explanation of the method of use of the present disclosure for physicians or other users. The instruction provides description for the method of detection of the present disclosure, how to use a diagnostic drug, or instruction for administration of a medicament or the like. The instruction may have a description instructing administration to the eye as a site of administration (e.g., by eye drop instillation, ophthalmic ointment, injection, etc.). The instruction is prepared in accordance with a format specified by a regulatory authority of the country in which the present disclosure is practiced (e.g., Ministry of Health, Labour and Welfare in Japan, Food and Drug Administration (FDA) in the U.S., or the like), with an explicit description showing approval by the regulatory authority. An instruction is a so-called package insert. An instruction is generally provided in, but not limited to, paper media. An instruction can also be provided in a form such as electronic media (e.g., web sites provided on the Internet or emails).

One embodiment can provide an eye drop comprising nicotinamide mononucleotide (NMN), wherein the nicotinamide mononucleotide (NMN) is contained at a concentration of 0.001 μg/ml or greater, 0.01 μg/ml or greater, 0.1 μg/ml or greater, 1 μg/ml or greater, 10 μg/ml or greater, 100 μg/ml or greater, 200 μg/ml or greater, 500 μg/ml or greater, 1 mg/ml or greater, 2 mg/ml or greater, 5 mg/ml or greater, 10 mg/ml or greater, or 50 mg/ml or greater.

One embodiment can provide an eye drop comprising nicotinamide riboside (NR), wherein the nicotinamide riboside (NR) is contained at a concentration of 0.001 μg/ml or greater, 0.01 μg/ml or greater, 0.1 μg/ml or greater, 1 μg/ml or greater, 10 μg/ml or greater, 100 μg/ml or greater, 200 μg/ml or greater, 500 μg/ml or greater, 1 mg/ml or greater, 2 mg/ml or greater, 5 mg/ml or greater, 10 mg/ml or greater, or 50 mg/ml or greater.

One embodiment can provide an ophthalmic ointment comprising nicotinamide mononucleotide (NMN), wherein the nicotinamide mononucleotide (NMN) is contained at a concentration of 0.001 μg/ml or greater, 0.01 μg/ml or greater, 0.1 μg/ml or greater, 1 μg/ml or greater, 2 μg/ml or greater, 5 μg/ml or greater, 10 μg/ml or greater, 50 μg/ml or greater, 100 μg/ml or greater, 200 μg/ml or greater, or 500 μg/ml or greater.

One embodiment can provide an ophthalmic ointment comprising nicotinamide riboside (NR), wherein the nicotinamide riboside (NR) is contained at a concentration of 0.001 μg/ml or greater, 0.01 μg/ml or greater, 0.1 μg/ml or greater, 1 μg/ml or greater, 2 μg/ml or greater, 5 μg/ml or greater, 10 μg/ml or greater, 50 μg/ml or greater, 100 μg/ml or greater, 200 μg/ml or greater, or 500 μg/ml or greater.

Use of a formulation with a concentration at a predetermined amount or greater in accordance with the dosage form can be advantageous for delivery to a meibomian gland, which is at the back of an eyelid, unlike delivery on the eye ball.

(Dose)

In one embodiment, utilization methods of the present disclosure include, for example, an eye drop, but the methods are not limited thereto. Examples thereof include modes of administration (administration methods and dosage forms) such as ophthalmic ointment, injection into the anterior chamber, impregnation into a sustained release agent, subconjunctival injection, and systemic administration (oral administration and intravenous injection).

The concentration of NMN or NR used in the present disclosure is generally about 0.001 to 1000 μM (μmol/l), preferably about 0.01 to 300 μM, more preferably about 0.03 to 100 μM, and still more preferably about 0.1 to about 30 μM. For example, other concentration ranges are generally 0.01 nM to 100 μM, about 0.1 nM to 100 μM, about 0.001 to 100 μM, about 0.01 to 75 μM, about 0.05 to 50 μM, about 1 to 10 μM, about 0.01 to 10 μM, about 0.05 to 10 μM, about 0.075 to 10 μM, about 0.1 to 10 μM, about 0.5 to 10 μM, about 0.75 to 10 μM, about 1.0 to 10 μM, about 1.25 to 10 μM, about 1.5 to 10 μM, about 1.75 to 10 μM, about 2.0 to 10 μM, about 2.5 to 10 μM, about 3.0 to 10 μM, about 4.0 to 10 μM, about 5.0 to 10 μM, about 6.0 to 10 μM, about 7.0 to 10 μM, about 8.0 to 10 μM, about 9.0 to 10 μM, about 0.01 to 50 μM, about 0.05 to 5.0 μM, about 0.075 to 5.0 μM, about 0.1 to 5.0 μM, about 0.5 to 5.0 μM, about 0.75 to 5.0 μM, about 1.0 to 5.0 μM, about 1.25 to 5.0 μM, about 1.5 to 5.0 μM, about 1.75 to 5.0 μM, about 2.0 to 5.0 μM, about 2.5 to 5.0 μM, about 3.0 to 5.0 μM, about 4.0 to 5.0 μM, about 0.01 to 3.0 μM, about 0.05 to 3.0 μM, about 0.075 to 3.0 μM, about 0.1 to 3.0 μM, about 0.5 to 3.0 μM, about 0.75 to 3.0 μM, about 1.0 to 3.0 μM, about 1.25 to 3.0 μM, about 1.5 to 3.0 μM, about 1.75 to 3.0 μM, about 2.0 to 3.0 μM, about 0.01 to 1.0 μM, about 0.05 to 1.0 μM, about 0.075 to 1.0 μM, about 0.1 to 1.0 μM, about 0.5 to 1.0 μM, about 0.75 to 1.0 μM, about 0.09 to 35 μM, or about 0.09 to 3.2 μM, more preferably about 0.01 to 10 μM, about 0.1 to 3 μM, or about 0.1 to 1.0 μM, but the concentration range is not limited thereto.

When used as an eye drop, the concentration of the formulation can be determined based on about 1 to 10000-fold, preferably about 100 to 10000-fold, such as about 1000-fold of the effective concentration described above while taking dilution with lacrimal fluid or the like into consideration as well as toxicity. The concentration can also be set to a concentration in excess thereof. The concentration is, for example, about 0.01 μM (μmol/l) to 1000 mM (mmol/l), 0.03 μM to 1000 mM, about 0.1 μM to 300 mM, about 0.3 μM to 300 mM, about 1 μM to 100 mM, about 3 μM to 100 mM, about 10 μM to 100 mM, about 30 μM to 100 mM, about 0.1 μM to 30 mM, about 0.3 μM to 30 mM, about 1 μM to 30 mM, about 3 μM to 30 mM, about 1 μM to 10 mM, about 3 μM to 10 mM, about 10 μM to 1 mM, about 30 μM to 1 mM, about 10 μM to 10 mM, about 30 μM to 10 mM, about 100 μM to 10 mM, about 300 μM to 10 mM, about 10 μM to 100 mM, about 30 μM to 300 mM, about 100 μM to 300 mM, or about 300 μM to 300 mM, and can be about 1 mM to 10 mM or about 1 mM to 100 mM. The concentration range can be determined by appropriately combining these upper limits and lower limits.

The effect amount of the medicament of the present disclosure, which is effective in treating a specific disease, disorder or condition, can vary depending on the property of the disorder or condition, but can be determined with a standard clinical technology based on the descriptions herein by those skilled in the art. Furthermore, identification of the optimal dosage range can be optionally assisted by using an in vitro assay. Since the precise dose to be used in a compound can vary depending on the route of administration or severity of a disease or disorder, the dose should be determined in accordance with the judgment of a physician or status of each patient. The dosage is not particularly limited, but can be, for example, 0.001, 1, 5, 10, 15, 100, or 1000 mg/kg body weight per administration or within a range of two of such values. The dosing interval is not particularly limited, but can be, for example, 1, 2, or 4 doses every 1, 7, 14, 21, or 28 days, or 1, 2, or 4 doses for every number of days in the range between any two of such values. The dosage, number of doses, dosing interval, dosing period, and dosing method can be appropriately selected depending on the patient's age or body weight, symptom, mode of administration, target organ, or the like. For example, the composition of the present disclosure can be used as an eye drop. Further, a therapeutic drug preferably comprises an active ingredient at a therapeutically effective amount or an amount effective to exert a desired action. The effective dose can be estimated from a dose-response curve obtained from an in vitro or animal model test system.

Administration of an active ingredient at a predetermined amount of higher can be advantageous for delivery to a meibomian gland, which is at the back of an eyelid, unlike delivery on the eye ball.

(Therapeutic Method and Prophylactic Method)

The present disclosure can provide a method of improving activity of Hsd3b6 or a homolog thereof or treating or preventing a disease, disorder, or symptom associated with activity of Hsd3b6 or a homolog thereof. The method can comprise administering nicotinamide mononucleotide (NMN) and/or nicotinamide riboside (NR) to a subject.

The method can optionally comprise measuring activity of Hsd3b6 or a homolog thereof in a subject. One embodiment can provide a method of administering NMN and/or NR when the activity of Hsd3b6 or a homolog thereof in a subject is measured and the activity is diminished.

The present disclosure can provide a method for improving a function of a meibomian gland or treating or preventing a disease, disorder, or symptom accompanied by meibomian gland dysfunction. The method can comprise administering nicotinamide mononucleotide (NMN) and/or nicotinamide riboside (NR) to a subject.

The present disclosure can provide a method of enhancing testosterone generation in a meibomian gland. The method can comprise administering nicotinamide mononucleotide (NMN) and/or nicotinamide riboside (NR) to a subject.

The method can optionally comprise inspecting a meibomian gland function of a subject. One embodiment can provide a method of administering NMN and/or NR when the meibomian gland function of the subject is inspected and the function is diminished. The meibomian gland function can be inspected by, for example, a slit-lamp examination, Meibography, Confocal microscopy, DR-1, mucocutaneous junction, meibometer, Lipid chemistry, amount of evaporation, BUT, or a subjective symptom questionnaire.

(General Technology)

The molecular biological methodology, biochemical methodology, and microbiological methodology used herein are well known and conventionally used in the art, which are described, for example, in Sambrook J. et al. (1989). Molecular Cloning: A Laboratory Manual, Cold Spring Harbor and its 3rd Ed. (2001); Ausubel, F. M. (1987). Current Protocols in Molecular Biology, Greene Pub. Associates and Wiley-Interscience; Ausubel, F. M. (1989). Short Protocols in Molecular Biology: A Compendium of Methods from Current Protocols in Molecular Biology, Greene Pub. Associates and Wiley-Interscience; Innis, M. A. (1990). PCR Protocols: A Guide to Methods and Applications, Academic Press; Ausubel, F. M. (1992). Short Protocols in Molecular Biology: A Compendium of Methods from Current Protocols in Molecular Biology, Greene Pub. Associates; Ausubel, F. M. (1995). Short Protocols in Molecular Biology: A Compendium of Methods from Current Protocols in Molecular Biology, Greene Pub. Associates; Innis, M. A. et al. (1995). PCR Strategies, Academic Press; Ausubel, F. M. (1999). Short Protocols in Molecular Biology: A Compendium of Methods from Current Protocols in Molecular Biology, Wiley, and annual updates; Sninsky, J. J. et al. (1999). PCR Applications: Protocols for Functional Genomics, Academic Press, Gait, M. J. (1985). Oligonucleotide Synthesis: A Practical Approach, IRL Press; Gait, M. J. (1990). Oligonucleotide Synthesis: A Practical Approach, IRL Press; Eckstein, F. (1991). Oligonucleotides and Analogues: A Practical Approach, IRL Press; Adams, R. L. et al. (1992). The Biochemistry of the Nucleic Acids, Chapman & Hall; Shabarova, Z. et al. (1994). Advanced Organic Chemistry of Nucleic Acids, Weinheim; Blackburn, G. M. et al. (1996). Nucleic Acids in Chemistry and Biology, Oxford University Press; Hermanson, G. T. (I996). Bioconjugate Techniques, Academic Press, Bessatsu Jikken Igaku [Experimental Medicine, Supplemental Volume], “Idenshi Donyu & Hatsugen Kaiseki Jikken Ho [Experimental Methods for Transgenesis & Expression Analysis]”, Yodosha, 1997, or the like. Relevant portions thereof (which may be the entire document) are incorporated herein by reference.

Reference literatures such as scientific literatures, patents, and patent applications cited herein are incorporated herein by reference to the same extent that the entirety of each document is specifically described.

The present disclosure has been described while showing preferred embodiments to facilitate understanding. While the present disclosure is described hereinafter based on the Examples, the above descriptions and the following Examples are provided for the sole purpose of exemplification, not limitation of the present disclosure. Thus, the scope of the present disclosure is not limited to the embodiments and Examples that are specifically described herein and is limited only by the scope of claims.

### EXAMPLES

Examples of the present disclosure are described hereinafter. Biological samples or the like, where applicable, were handled in compliance with the standards enacted by the Ministry of Health, Labour and Welfare, Ministry of Education, Culture, Sports, Science and Technology, or the like and, where applicable, based on the Declaration of Helsinki or ethical codes prepared based thereon.

### Example 1: Atrophy of Meibomian Gland Tissue Associated with Aging/Meibomian Gland Tissue in Hsd3b6 KO Mice

### [Summary]

This Example demonstrates atrophy of meibomian gland tissue associated with aging and the change in meibomian gland tissue in Hsd3b6 KO mice.

[Materials and Methods]

### (1) Whole Mount Meibomian Glands Herxheimer's Staining Protocol

Meibomian gland tissue in mice was stained in accordance with the following protocol.

As a preparation method of 100 mL of staining solution, a bottle of stock solution prepared by dissolving Sudan IV to saturate in 100% ethanol was left standing in advance, and 70 mL of as much of the supernatant, 20 mL of 10% NaOH, and 10 mL of ultrapure water were mixed, tumbled for 5 minutes, and centrifuged to allow powder to precipitate. The supernatant was filtered with a 0.45 μm filter and used as a staining solution.

The cervical vertebrae of mice was dislocated, and skin of the head including meibomian glands was sampled. The sample was immersed in 4% paraformaldehyde while keeping the sample flat with a drawing pin, and immobilized using a rotator at 4° C. for 24 hours. The periphery of meibomian glands was trimmed. The meibomian glands were immersed in 50% ethanol by using a rotator at room temperature for 10 minutes, then immersed for 20 minutes in 70% ethanol, and immersed in a staining solution using a rotator at room temperature for 24 hours in a falcon tube. The meibomian glands were then immersed for 10 minutes in 70% ethanol, and washing was repeated several times to confirm that the meibomian glands were stained well. The meibomian glands were immersed for 24 hours in amino alcohol in glycerin prepared by mixing 3.75 mL of 80% amino alcohol with 8.25 mL glycerin. The meibomian glands were placed on a microscope slide and encapsulated with glycerin jelly. Glycerin jelly was prepared by first mixing 10 g of gelatin with 60 ml of ultrapure water and 70 ml of glycerin, expanding the gelatin, and adding glycerin after about one hour and mixing while heating.

(2) Analysis of Area of Meibomian Gland Stained with Sudan IV

Post-staining area analysis was performed by using Photoshop and ImageJ and determining a threshold value of color intensity while looking at and comparing with a stain image based on the area of portions with an intensity at or above a given threshold value.

### (3) Mouse

A floxed mouse in which the Exon 2 region including a start codon of an Hsd3b6 gene of a mouse is flanked by loxP was prepared. The mouse was crossbred with a CAG-Cre transgenic mouse to prepare an Hsd3b6 KO mouse which could no longer express Hsd3b6.

A test was conducted after raising a two month old mouse for 10 days under normal conditions (normal humidity condition) for free-feeding/drinking environment in a 12 hour bright and 12 hour dark cycle with a humidity of 40 to 60% and room temperature of 25° C.

A 24 month old mouse was a 78 week old (18 month old) male C57BL/6J mouse purchased from OrientalBioService, Inc., which was raised for 6 months under normal conditions (normal humidity conditions). A 6 month old C57BL/6J mouse was a 6 week old mouse purchased and raised to 6 month old under normal conditions (normal humidity conditions).

[Results]

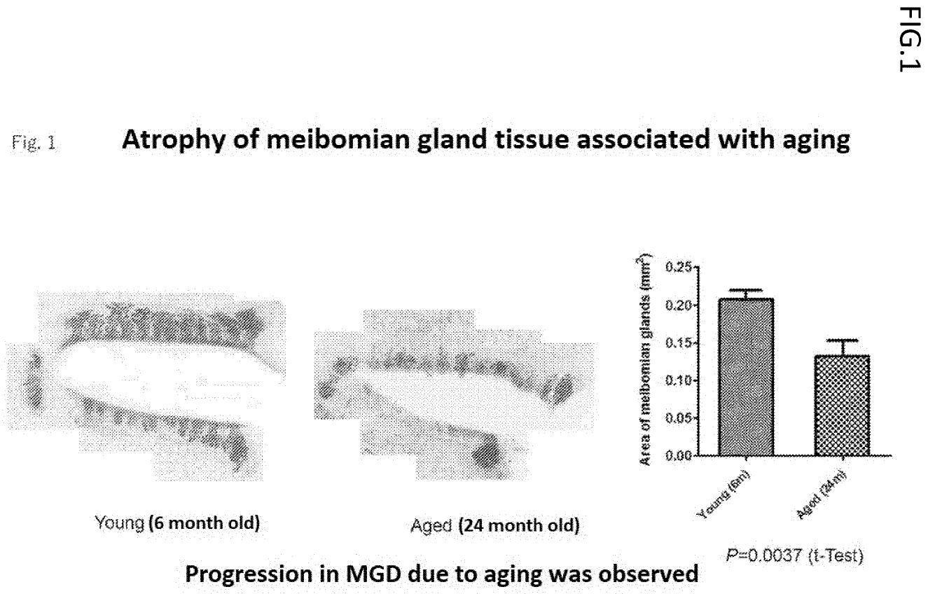

Meibomian gland tissue was stained in a 6 month old mouse and a 24 month old mouse in accordance with the protocol described above, and the stained areas were compared. The results are shown in FIG. 1. Atrophy in meibomian gland tissue and progression in meibomian gland dysfunction (MGD) due to aging were observed (N=7).

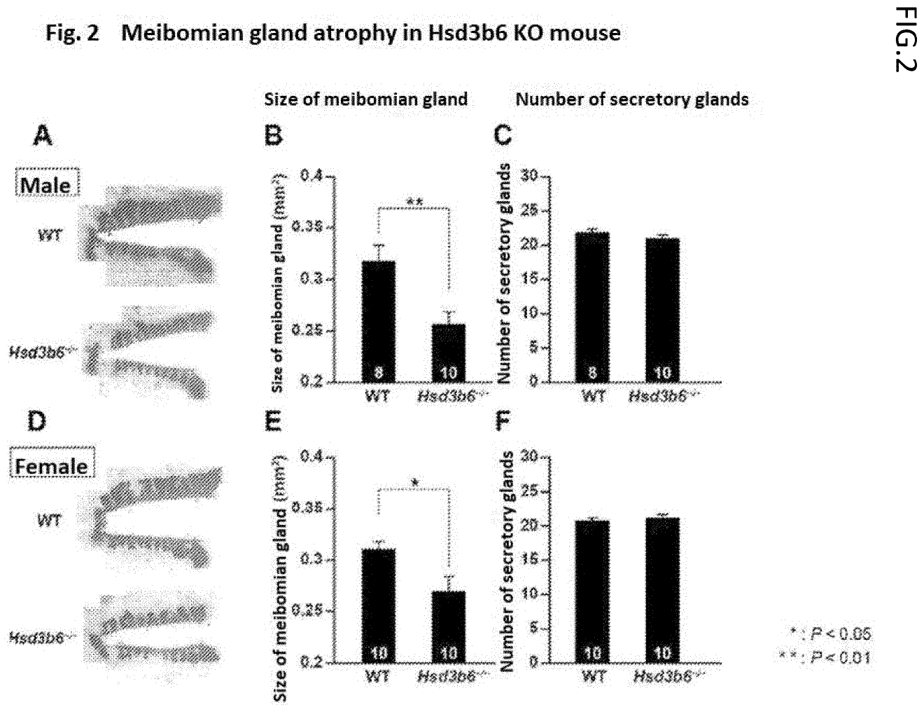

FIG. 2 shows the size of meibomian gland tissue and the number of secretory ducts in a 2 month old Hsd3b6 KO mouse and wild-type mouse. Just like in aged mice, Hsd3b6 KO mice were also found to exhibit meibomian gland atrophy. Since there is no difference in the number of secretory ducts due to genotype, it was found that meibomian gland atrophy is not an abnormality in the process of generating ducts.

### Example 2: Experiment of Eye Drop Instillation of NMN or NR into a 1.5 Year Old Wild-Type Mouse for 2 Weeks

[Summary]

This Example demonstrates the change in Hsd3b6 enzymatic activity and proliferative activity of meibomian gland acinar basal cells in meibomian glands due to eye drop instillation of NMN or NR for 2 weeks into 1.5 year old wild-type mice.

[Materials and Methods]

### (1) The Following is the Summary of the Eye Drop Instillation Experiment.

TABLE 1

| | | | Route of | Dosing | Number of | |
|---|---|---|---|---|---|---|
| Animal | Compound | Dosage | administration | period | animals | Experiment |
| WT mice | Vehicle | — | Eye drop | 2 weeks | 5 | Enzymatic activity (steroid |
| (1.5 year | | | instillation (only | | | synthesis activity) |
| old) | | | right eye) | | 5 | BrdU incorporation assay |
| | | | | | | (cell proliferative activity) |
| | NMN | 5% (5 | | | 6 | Enzymatic activity (steroid |
| | | μL/eye, 6 | | | | synthesis activity) |
| | | times/day) | | | 6 | BrdU incorporation assay |
| | | | | | | (cell proliferative activity) |
| | NR | 5% (5 | | | 5 | Enzymatic activity (steroid |
| | | μL/eye, 6 | | | | synthesis activity) |
| | | times/day) | | | 6 | BrdU incorporation assay |
| | | | | | | (cell proliferative activity) |

### (2) Measurement of Meibomian Gland Hsd3b6 Enzymatic Activity

The meibomian gland Hsd3b6 enzymatic activity in mice was measured in accordance with the following protocol.

Preliminary Preparation

Hank's balanced salt solution (HBSS) was bubbled with 95% CO₂/5% 02 for 1 hour or more, and dispensed in 2 mL tubes at 200 μL each. The lid was closed while filled with 95% CO₂/5% 02. HBSS was warmed with a 37° C. water bath. A reaction incubator was warmed to 37° C., and a micro tube mixer was installed therein.

Startup of Measuring Equipment

The main power of a flow liquid scintillation counter (Perkin Elmer, Radiomatic 625TR) was turned on, and the main power of HPLC (Waters, 2795 Separations Module) was turned on to perform setup. Mobile phase A was replaced with water, and mobile phase B was replaced with acetonitrile. Once a column (Kanto Chemical, LiChroCART 250-4, LiChrosphere 100RP-18, particle size: 5 μm, 4 mm I.D.×250 mm) was installed, 40% acetonitrile (ACN) (A: 60%, B: 40%) was flown at 0.7 mL/min. The column oven temperature at this time was set to 40° C.

Purification of ³H-Pregnenolone

³H-DHEA was added to 1 mL of 40% ACN contained in a 2 mL tube, and the mixture was tapped. A microtube was set on an HPLC holder, and single administration was selected. The peak of contaminant produced after 22 minutes was not collected. Once the peak of contaminant decreased at about 23 minutes, the peak of ³H-DHEA was then manifested, so that collection into a glass centrifuge tube was started after reaching ⅓ of c.p.m. of the value of the peak. About 2.5 mL was collected in total from 4 minutes of collection. The liquid inside the glass centrifuge tube was stirred with a vortex. 25 μL was subjected to the liquid scintillation counter, and 25 μL was added to 3 mL of clear sol. After confirming that a small amount of N₂ was being discharged from an exhaust nozzle, the liquid inside the glass centrifuge tube was exsiccated with nitrogen at 75° C. using Taitec's incubator.

Preparation of Substrate

After cooling the ³H-DHEA exsiccated with nitrogen to room temperature, 1 μL/4,000 c.p.m. (value from measuring 25 μL with a liquid scintillation counter) of propylene glycol was added, and the ³H-DHEA was dissolved by vortexing for 5 minutes.

4 μL/4,000 c.p.m. of PBS was added. The mixture was vortexed. 10 μL was subjected to a liquid scintillation counter to confirm that the count is above 400000 c.p.m. The mixture was diluted with PBS:propylene glycol=4:1. 400000 c.p.m./10 μL hot solution (1), 87 μM DHEA in 10% EtOH/90% D-PBS (2), 1 mM Dutasteride in 50% DMSO/50% DW (3), and 1 mM Fadrozole in 50% DMSO/50% DW (4) were prepared and mixed at a ratio of (1):(2):(3):(4)=5:5:1:1 as a solution of a substrate. This was warmed with a 37° C. water bath prior to use.

Enzymatic Reaction and Extraction

A meibomian gland of the upper eyelid of one of the eyes was cut out and placed on weighing paper, with the side exposing the meibomian gland on top. The sample was chopped up into 200 μm thickness with a tissue chopper. The chopped meibomian gland was transferred into HBSS. The substrate solution was gently vortexed, and 24 μL was added to HBSS. The mixture was pipetted.

The mixture was incubated for 30 minutes while shaking with a micro tube mixer installed inside a 37° C. incubator. After 30 minutes, the mixture was centrifuged at room temperature for 1 minute at 700 g, and the meibomian gland tissue was allowed to precipitate. The entire amount of HBSS was transferred into a test tube to which 2 mL of ethyl acetate was dispensed. The mixture was vortexed for 10 seconds. When the reaction was stopped, the mixture was immediately transferred onto ice.

200 μL of HBSS warmed to 37° C. was added to a 2 mL tube with a meibomian gland remaining therein. After gently vortexing the substrate solution, 24 μL was added to HBSS, and the mixture was pipetted. 2 μL of 100 mM NAD⁺ in D-PBS was added. The mixture was incubated for 30 minutes while shaking with a micro tube mixer installed inside a 37° C. incubator. The mixture was centrifuged at room temperature for 1 minute at 700 g, and the meibomian gland tissue was allowed to precipitate. The entire amount of HBSS was transferred into a test tube to which 2 mL of ethyl acetate was dispensed. The mixture was vortexed for 10 seconds. When the reaction was stopped, the mixture was immediately transferred onto ice.

Steroid Extraction

The mixture was centrifuged at room temperature for 10 minutes at 1500 rpm. 1.6 mL of a layer of ethyl acetate at the top layer was collected and exsiccated with nitrogen at 75° C. When the liquid was completely evaporated, 500 μL of 40% ACN was added. The mixture was vortexed for 3 minutes. The liquid was transferred to MILLIPORE Ultra free-MC installed on a 2 mL tube and centrifuged at room temperature for 2 minutes at 12000 g. A glass centrifuge tube was prewashed with 500 μL of 40% ACN and vortexed for 2 minutes. After passing through the same column's filter, the mixture was centrifuged at room temperature for 4 minutes at 12000 g. The elute was mixed by tapping, and 10 μL was added to 3 mL of clear sol. The mixture was subjected to a liquid scintillation counter (measurement of recovery rate).

Measurement

The column was equilibrated under the initial conditions (until the baseline and pressure stabilized). Sample sets were created in the same number as the number of samples to be measured. Since analysis does not stabilize for the first column, 40% ACN was allowed to flow as a sample. The lid of a 2 mL tube to be measured was opened. The tube was covered with a thinly stretched Parafilm and set up for HPLC. Continuous analysis was started.

HPLC conditions (³H-DHEA)
Flow rate: 0.7 mL/min
Column temperature: 40° C.

### Gradient

0 to 30 min: 40 to 50% ACN (0.3%/min)

### 30 to 35 min: 70% ACN

### 35 to 40 min: 100% ACN

40 to 50 min: 100 to 40% ACN (6%/min)

### 50 to 60 min: 40% ACN

Analysis

A chromatograph was opened with ProFSA. Smoothing was performed by setting the SDA (smoothing function) level to 8. The peaks were displayed with Find Peaks and Locate Peaks. The Report Preview was copied and pasted onto Word. Data was retrieved to deduce the enzymatic activity by the following formula.

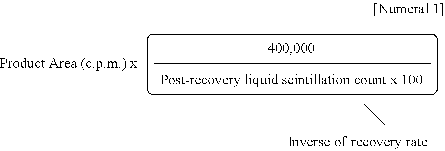

### (3) Cell Proliferation Assay Through BrdU Incorporation Staining

BrdU (100 mg/kg body weight, Sigma-Aldrich) was intraperitoneally injected 30 minutes prior to harvesting eyelid tissue from a mouse. An eyelid comprising meibomian glands was immobilized with 4% PFA and embedded in paraffin. Antigens were activated by pressurizing a 5 μm thick segment for 2.5 minutes in 10 mM sodium citrate buffer (pH 6.0). After incubating the segment for 24 hours at 4° C. by using an anti-BrdU antibody (1:1000 dilution; Rockland Inc.), the immune response was made visible with 3,3′-diaminobenzidine (DAB) using secondary antibody Envision+ System—HRP Labelled Polymer Anti-Rabbit (Dako). For each tissue segment, BrdU positive cells in the basal cell layer of a meibomian gland were counted on a digital microscope picture captured at 20× magnification. The length of the outer circumference of a meibomian gland acinus was measured with ImageJ software, and the total BrdU positive cell count was normalized with the length of the outer circumference. The BrdU positive cells were counted by using 16 segments per individual.

### [Results]

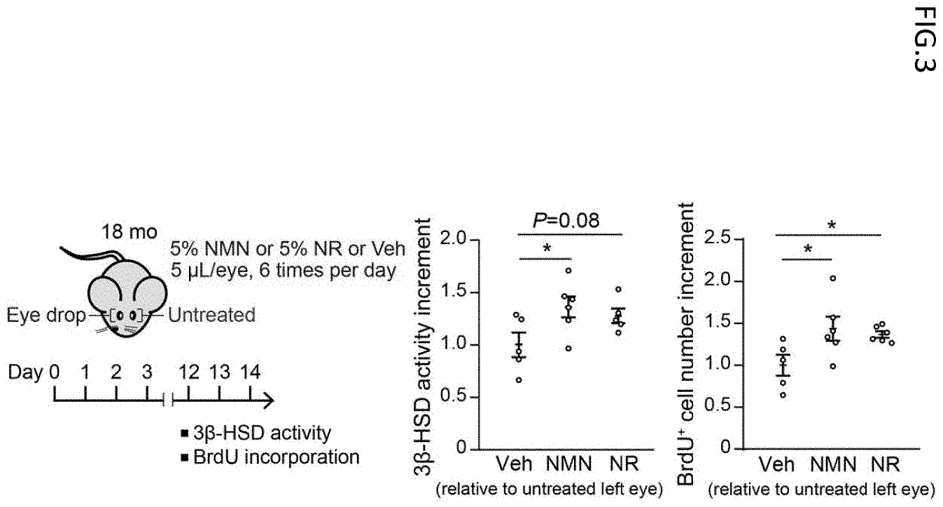

FIG. 3 shows the change in Hsd3b6 enzymatic activity in a meibomian gland and change in the BrdU positive cell count in meibomian gland basal cells due to eye drop instillation of NMN, NR, or a solvent thereof (Vehicle, phosphate buffered saline). Hsd3b6 enzymatic activity and BrdU positive cell count increased significantly due to NMN eye drop instillation. Similar increase in Hsd3b6 enzymatic activity was observed, and BrdU positive cell count also increased significantly in the NR eye drop instillation group. Specifically, it can be understood that Hsd3b6 enzymatic activity increases topically at a meibomian gland and the proliferative activity of meibomian gland basal cells increases due to NMN or NR eye drop instillation.

### Example 3: 3 Month Experiment of NMN or NR Eye Drop Instillation into 1.75 Year Old Wild-Type Mouse

[Summary]

This Example demonstrates the effect of 3 month NMN or NR eye drop installation into a 1.75 year old wild-type mouse on the meibomian gland tissue volume.

[Materials and Methods]

### (1) The Following is the Outline of the Eye Drop Instillation Experiment.

TABLE 2

| | | | Route of | Dosing | Number of | |
|---|---|---|---|---|---|---|
| Animal | Compound | Dosage | administration | period | animals | Experiment |
| WT mouse | Vehicle | — | Eye drop | 3 months | 8 | Lipid stained |
| (1.75 year | NMN | 5% (5 μL/eye, 4 | instillation | | 8 | tissue observation |
| old) | | times/day) | (only right eye) | | | (meibomian gland |
| | NR | 5% (5 μL/eye, 4 | | | 10 | volume evaluation) |
| | | times/day) | | | | |

### (2) Meibomian Gland Tissue in a Mouse was Stained Through the Whole Mount Meibomian Glands Herxheimer's Staining in the Same Manner as the Approach in Example 1.

[Results]

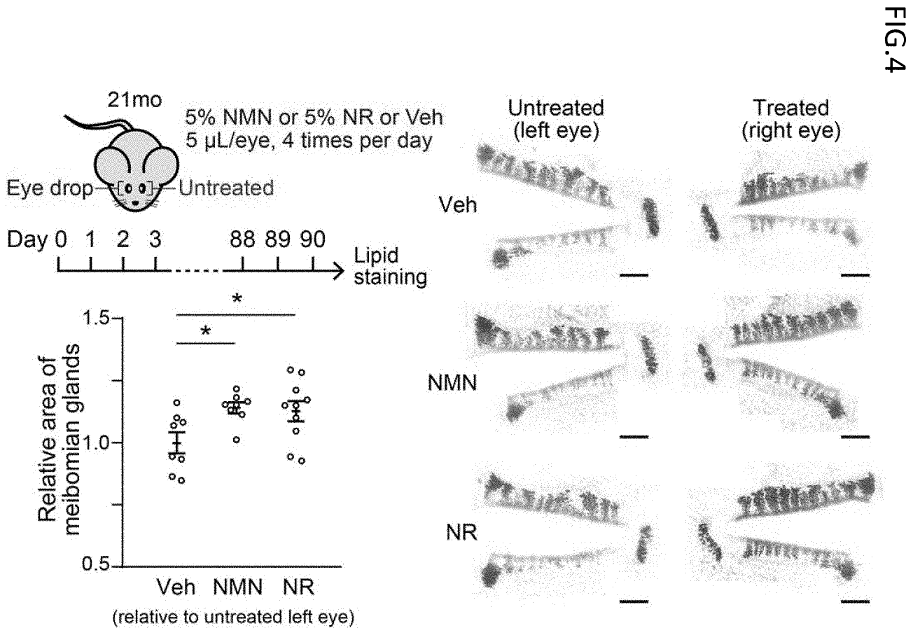

FIG. 4 shows the change in meibomian gland volume due to eye drop instillation of NMN, NR, and solvent thereof (Vehicle, phosphate buffered saline). A significant increase in the meibomian gland volume was observed for both NMN eye drop instillation group and NR eye drop instillation group.

### Example 4: 3 Month Experiment of NMN or NR Eye Drop Instillation into 1.75 Year Old Wild-Type Mouse

[Summary]

This Example demonstrates the effect of 3 month NMN or NR eye drop instillation into 1.75 year old wild-type mouse on lipid components in meibomian glands.

[Materials and Methods]

### (1) The Eye Drop Instillation Experiment is Performed in the Same Manner as Example 3.

(2) Harvest of Lipids from Mouse Meibomian Glands and Lipid Analysis

Meibomian glands are harvested from an eyelid of a euthanized mouse. Two meibomian glands harvested from one of the eyes of each animal are each placed in a glass vial containing 1 mL of chloroform:methanol=2:1 (v/v) solvent mixture for lipid extraction, and lipids are extracted. The extract is relocated into a new vial. The extract can be stored for several months at −20° C. or lower by evaporating the solution under nitrogen. The extract is then dissolved in a suitable solvent, and lipid composition is analyzed by APCI.

[Results]

Lipid components in a meibomian gland are normalized in NMN or NR eye drop instillation groups compared to a control Vehicle (phosphate buffered saline) eye drop instillation group.

### Example 5: Quantification of Testosterone in Meibomian Gland

[Materials and Methods]

After homogenizing a meibomian gland resected from an eyelid with methanol/water (75:25, v/v), steroid contained in the homogenate was extracted into dichloromethane by using Isolute SLE+ cartridge (Biotage) and evaporated under nitrogen to obtain a residue. The residue was dissolved in 50 μL of methanol/5% acetic acid solution containing an Amplifex Keto reagent (SCIEX) with a concentration of 10 mg/mL and incubated for 1 hour at room temperature. The testosterone concentration was then found by mass spectrometry using a liquid chromatography electrospray ionization tandem mass spectrometer (QTRAP 4500 LC-MS/MS System; SCIEX) (derivative SRM transition: 403-.164), and normalized by the wet weight of the extracted meibomian gland.

[Results]

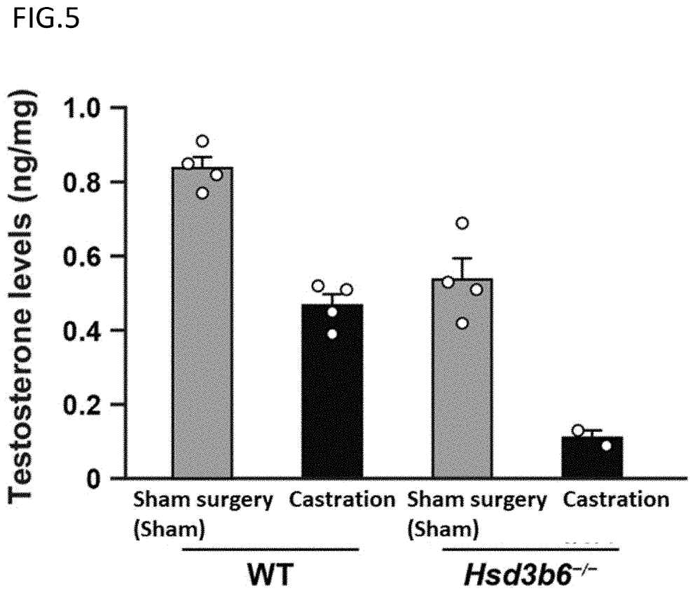

FIG. 5 shows the amount of testosterone in meibomian gland tissue of wild-type and Hsd3b6 deficient mice. The testosterone contained topically in meibomian glands decreased about 50% in Hsd3b6 deficient mice compared to wild-type mice. Furthermore, testosterone detected topically at meibomian glands decreased to 10% or less in Hsd3b6 knockout mice with the gonad (testicle) resected. It can be understood therefrom that not only circulating testosterone supplied from the testes, but also the same level of testosterone production topical to the meibomian gland contribute at the meibomian glands.

Since Hsd3b6 enzymatic activity is increased by eye drop instillation of NMN or NR, it is understood that production of testosterone which is known to promote lipid production of meibomian glands is promoted, and lipid component composition in meibomian gland tissue is normalized.

(Note)

As described above, the present disclosure is exemplified by the use of its preferred embodiments. However, it is understood that the scope of the present disclosure should be interpreted solely based on the Claims. It is also understood that any patent, any patent application, and any references cited herein should be incorporated herein by reference in the same manner as the contents are specifically described herein. The present application claims priority to Japanese Patent Application No. 2019-117642 filed on Jun. 25, 2019 with the Japan Patent Office. The entire content thereof is incorporated herein by reference in the same manner as if the contents are specifically described herein.

### INDUSTRIAL APPLICABILITY

The present disclosure can be used in the field of medicine, pharmaceutical products, healthcare, biology, biochemistry, and the like.

1. A method for improving a function of a meibomian gland in a subject, comprising administering to the subject a composition comprising nicotinamide mononucleotide (NMN) and/or nicotinamide riboside (NR).

2. The method of claim 1 wherein improving the function of the meibomian gland comprises increasing a number of meibomian gland acinar cells.

3. The method of claim 1 wherein improving the function of the meibomian gland comprises increasing lipid secretion from the meibomian gland.

4. The method of claim 1 which comprises treating or preventing a disease accompanied by dysfunction of a meibomian gland.

5. A method for treating or preventing meibomian gland dysfunction in a subject comprising administering to the subject a composition comprising nicotinamide mononucleotide (NMN) and/or nicotinamide riboside (NR).

6. The method of claim 5, wherein the meibomian gland dysfunction is accompanied by reduced meibum secretion.

7. The method of claim 5, wherein the meibomian gland dysfunction is accompanied by an inflammatory disease.

8. The method of claim 7, wherein the inflammatory disease comprises at least one of meibomitis, superficial or punctate keratitis, or blepharitis.

9. The method of claim 5, wherein the meibomian gland dysfunction is accompanied by excessive lipid accumulation in a duct.

10. The method of claim 5, wherein the meibomian gland dysfunction is accompanied by ocular discomfort, sensation of a foreign object, and/or sense of pressure.

11. The method of claim 4, wherein the disease accompanied by dysfunction of a meibomian gland is meibomitis, posterior blepharitis, keratitis, conjunctivitis, meibomitis-related keratoconjunctivitis, ocular pemphigoid, Sjogren's syndrome, Stevens-Johnson syndrome, graft-versus-host disease (GVHD), or visual function disorder.

12. The method of claim 2 which comprises treating or preventing a disease accompanied by dysfunction of a meibomian gland.

13. The method of claim 12, wherein the disease accompanied by dysfunction of a meibomian gland is meibomitis, posterior blepharitis, keratitis, conjunctivitis, meibomitis-related keratoconjunctivitis, ocular pemphigoid, Sjogren's syndrome, Stevens-Johnson syndrome, graft-versus-host disease (GVHD), or visual function disorder.

14. The method of claim 3 which comprises treating or preventing a disease accompanied by dysfunction of a meibomian gland.

15. The method of claim 14, wherein the disease accompanied by dysfunction of a meibomian gland is meibomitis, posterior blepharitis, keratitis, conjunctivitis, meibomitis-related keratoconjunctivitis, ocular pemphigoid, Sjogren's syndrome, Stevens-Johnson syndrome, graft-versus-host disease (GVHD), or visual function disorder.
